(19) United States
(12) Patent Application Publication
(10) Pub. No.: US 2021/0304863 A1
(43) Pub. Date: Sep. 30, 2021

(54) MEDICINAL DOSAGE COMPLIANCE SYSTEM

(71) Applicant: FirstEnergy Corp., Akron, OH (US)

(72) Inventors: Conner Patrick McCluskey, Tallmadge, OH (US); René Polin, Cleveland, OH (US); Jonathan Barrett Morgan, Westlake, OH (US); Nicholas Randolph Dauchot, Columbus, OH (US); Gabriel Jose Puerto, Bay Village, OH (US); Michael Christopher Tracz, Brecksville, OH (US)

(73) Assignee: FirstEnergy Corp., Akron, OH (US)

(21) Appl. No.: 17/214,202

(22) Filed: Mar. 26, 2021

Related U.S. Application Data
(60) Provisional application No. 63/000,105, filed on Mar. 26, 2020.

(51) Int. Cl. G16H 20/13; G16H 40/67; G16H 10/60; A61J 1/14

(52) U.S. Cl. CPC G16H 20/13; G16H 40/67; A61J 2205/60; A61J 1/1437; G16H 10/60

(57) ABSTRACT

A medicinal dosage compliance system may comprise a docking station, a container, and transactional information component. The docking station may comprise an interaction portion. The lockable container may be configured to hold medicine for a student, and the container may be configured to operably engage with the interaction portion. The transactional information component may be in operative communication with a computing device accessible by a user. The student and the container may be identified and associated with the identified student. Medication data may be communicated to the transactional information component indicative of a dosing event. The medication data may comprise at least a type of medication, a dosage amount of medicine to be taken by the student and a time when the medicine was taken by the student. A digital medical administration record for the identified person may be configured to record the medication data.

This application claims priority to provisional application having application No. 63/000,105 filed on Mar. 26, 2020, the entirety of which is incorporated herein by reference.

### BACKGROUND

Nearly everyone needs to take medication at some point in his or her lifetime. For some, medication becomes a part of daily life. For others, medication needs to be taken two or three times a day for a shorter duration, such as a week to ten days. One challenge facing individuals is compliance with taking medication. It can be difficult to remember to take all of a person's medication in a day and at the correct time intervals.

This medicinal compliance issue also applies to children. Often, for example, if a child is taking antibiotics, parents or guardians do their best to ensure the medication is taken as needed. However, it is easy to forget once children become asymptomatic. In other instances, children require continues medication, for example, to treat attention deficit disorder. In other instances, a child may simply have a headache and require a simple pain reliever such as ibuprofen or acetaminophen.

An additional complicating factor occurs when children are required to take medicine during school. State laws and regulations require vast amounts of paperwork from parents and health care providers. This information must be logged by a school. Next, a school nurse or other designated administrator must do their best to ensure children come to the nurse's office to take their medication, and then a record must be made. With numerous children coming at once, a nurse's office may become busy. Without a simple way to know if children are absent or simply late, a school nurse may find parts of his or her day inefficient as efforts are made to maintain compliance records.

There is a need for a simple to use and inexpensive system to securely store children's medication, easily track medicinal compliance, and maintaining an accurate medicinal transaction record.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

In one implementation, a medicinal dosage compliance system may comprise a docking station, a container, and transactional information component. The docking station may comprise an interaction portion. The lockable container may be configured to hold medicine for a student, and the container may be configured to operably engage with the interaction portion. The transactional information component may be in operative communication with a computing device accessible by a user. The student and the container may be identified and associated with the identified student. Medication data may be communicated to the transactional information component indicative of a dosing event. The medication data may comprise at least a type of medication, a dosage amount of medicine to be taken by the student and a time when the medicine was taken by the student. A digital medical administration record for the identified person may be configured to record the medication data.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

What is disclosed herein may take physical form in certain parts and arrangement of parts, and will be described in detail in this specification and illustrated in the accompanying drawings which form a part hereof and wherein:

FIG. 1 is a flow chart of the medicinal dosage compliance system.

FIG. 2 is a view of hardware for the system of FIG. 1.

FIG. 3 is a perspective view of a component of the medicinal dosage compliance system.

FIG. 4 is a perspective view of a component of the medicinal dosage compliance system.

FIG. 4A is a perspective view of a component of the medicinal dosage compliance system.

FIG. 4B is a perspective view of a component of the medicinal dosage compliance system.

FIG. 4C is a perspective view of a component of the medicinal dosage compliance system.

FIG. 4D is a perspective view of a component of the medicinal dosage compliance system.

FIG. 4E is a perspective view of a component of the medicinal dosage compliance system.

FIG. 5 is a perspective view of a component of the medicinal dosage compliance system.

FIG. 6 is a perspective view of a component of the medicinal dosage compliance system.

FIG. 7 is a perspective view of a component of the medicinal dosage compliance system.

FIG. 8A is a perspective view of a component of the medicinal dosage compliance system.

FIG. 8B is a perspective view of a component of the medicinal dosage compliance system.

FIG. 9 is a perspective view of a component of the medicinal dosage compliance system.

FIG. 10 is a flow chart for a process of the medicinal dosage compliance system.

FIG. 11 is a flow chart for a process of the medicinal dosage compliance system.

FIGS. 12A-12H are components of the medicinal dosage compliance system.

FIG. 13 is a flow chart for a process of the medicinal dosage compliance system.

FIGS. 14A-14E are components of the medicinal dosage compliance system.

FIGS. 15A-15C are components of the medicinal dosage compliance system.

FIGS. 16A-16D are components of the medicinal dosage compliance system.

FIG. 17 is a variety of implementations of the medicinal dosage compliance system.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

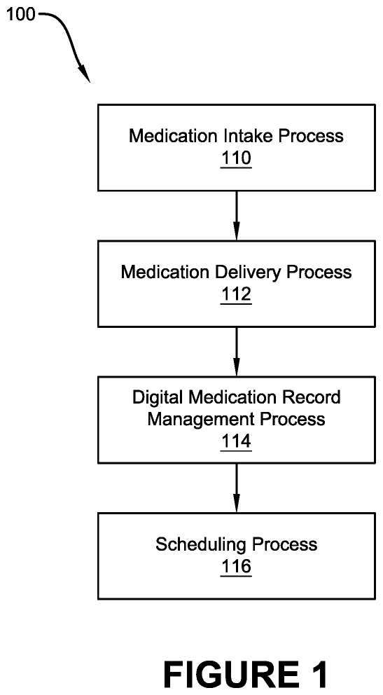

With reference to FIG. 1, a medicinal dosage compliance system 100 may comprise a new medication intake process 110, a medication delivery process 112, a digital medication record management process 114 and a scheduling process 116. Each process component may comprise hardware, software, mechanical, and electromechanical components. While the compliance system 100 describe herein discloses medical dosage compliance, any type of medical compliance, including without limitation vaccination administration and compliance.

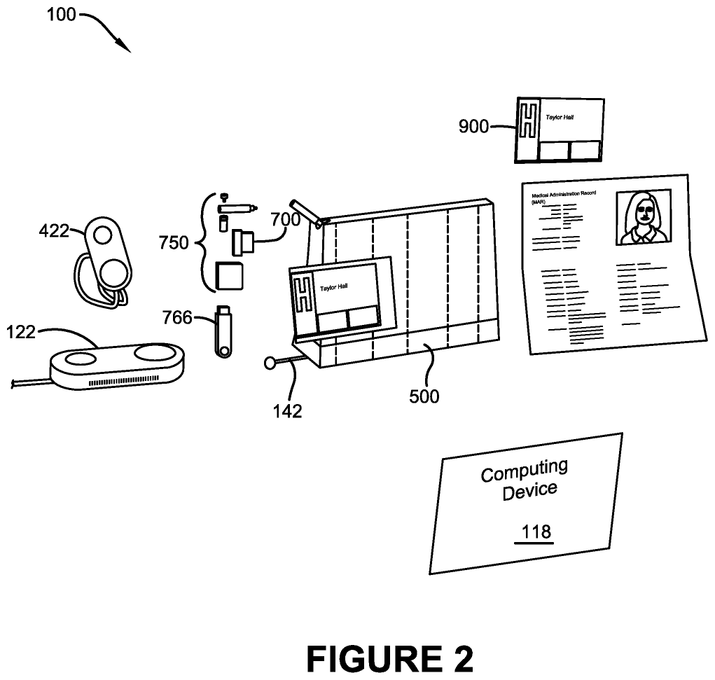

With reference to FIG. 2, multiple hardware components may be utilized for the medicinal dosage compliance system 100. A computing device 118 may be utilized. The computing device 118 may take any form, including without limitation, a desktop computer, a laptop computer, a tablet, smart phone, or other mobile device.

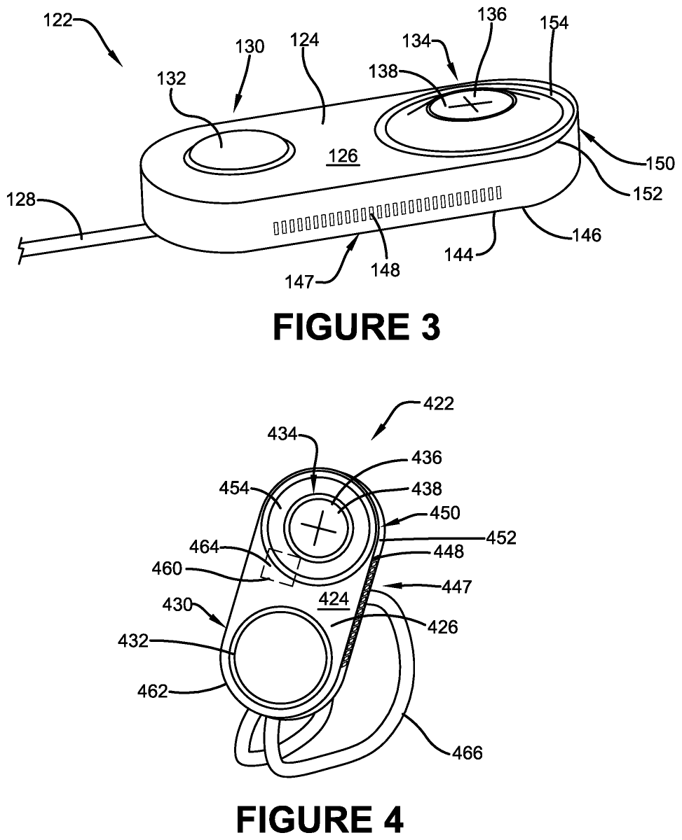

With reference to FIG. 3, a docking station 122 may be utilized for entering and reading biometric information. Biometric information may be entered or read for a user, which may comprise a student, nurse, administrator or other authorized user during administration of medication. The docking station 122 may be utilized for one or more of identification of a student, authentication of who the student is, and unlocking an associated container. This may occur in an office setting or a school setting, such as a school administrator's office. In one example, this may occur in the nurse's office. The docking station 122 may comprise a base 124, which may comprise an upper surface 126. A communication connector 128, such as a cord or cable, may be operably connected to the docking station 122 and the computing device 118. The communication connector 128 may be configured to transfer data between the office docking station 122 and the computing device 118. In another implementation, the docking station 122 may be configured to have a wireless connection 160. A local area network (e.g., a short-range or nearfield communication network) can be established between the docking station 122 and the computing device 118. As an example, the docking station 122 can comprise a Bluetooth module, and the computing device can comprise a separate Bluetooth module, which can be used to create a local Bluetooth connection with each other, for data sharing.

The docking station 122 may comprise a biometric reader 130. The biometric reader 130 may take any biometric input, including without limitation, fingerprints, iris recognition, retina recognition, palm veins, hand geometry, facial recognition, or DNA. In one nonlimiting implementation, the biometric reader may be a fingerprint reader 132. The fingerprint reader 132 may be sized to receive at least one digit. In other implementations, it may be sized to receive two or more digits. In other implementations, in addition to or instead of the biometric reader, any other type of reader may be utilized to receive data from any type of scanning identification to identify a user or a student.

With continued reference to FIG. 3, the docking station 122 may further comprise an interaction portion 134. The interaction portion 134 may also be disposed somewhere on the base 124. In one implementation, the interaction portion 134 may be disposed on the upper surface 126 of the base 124. The interaction portion 134 may be configured to comprise a communication tag reader 136. The interaction portion may be configured to also comprise an unlocking mechanism 138. In one implementation, the unlocking mechanism 138 may comprise a magnetic array that transfers motion to a locking mechanism 140 operably connected to a container 142.

With continued reference to FIG. 3, the docking station 122 may comprise feet 144, such as rubber feet 144 but not limited thereto. The feet 144 may be disposed on a lower surface 146 of the base 124. The feet 144 may be configured to provide stability on an underlying surface, such a desk or table, but not limited thereto. As multiple people, such as students, are repeatedly placing a biometric readable portion of their body in proximity to or on the biometric reader 130, the docking station 122 may be pushed or otherwise moved. The feet 144 may mitigate the amount of movement of the docking station 122 relative to the underlying surface upon which it is placed. The docking station 122 may also comprise an audible indicator 147 operably connected to the base 124. The audible indicator 147 may be configured to provide audible feedback to the user. In one nonlimiting example, the audible indicator may be a sound port 148. For example, the audible feedback may comprise one or more of success for entering data (e.g. biometric entry of a student or nurse or administrator), success for matching data (e.g. identification of a student or nurse or administrator), failure of entering data, failure for matching data or status of an interaction for the user, such as a student nurse or administrator. The audible indicator may make different sounds depending upon the action. By way of nonlimiting example, if the action is successful, the audible sound may be a higher pitch or a ding. However, if the action is unsuccessful, the audible sound may be a buzzer sound or other sound indicative of a negative result. A wide array of sounds may be chosen.

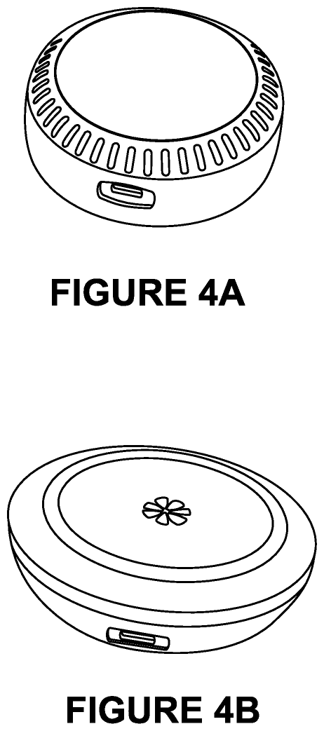

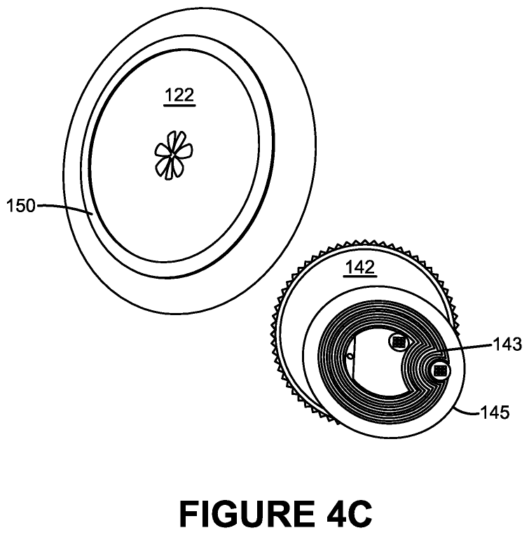

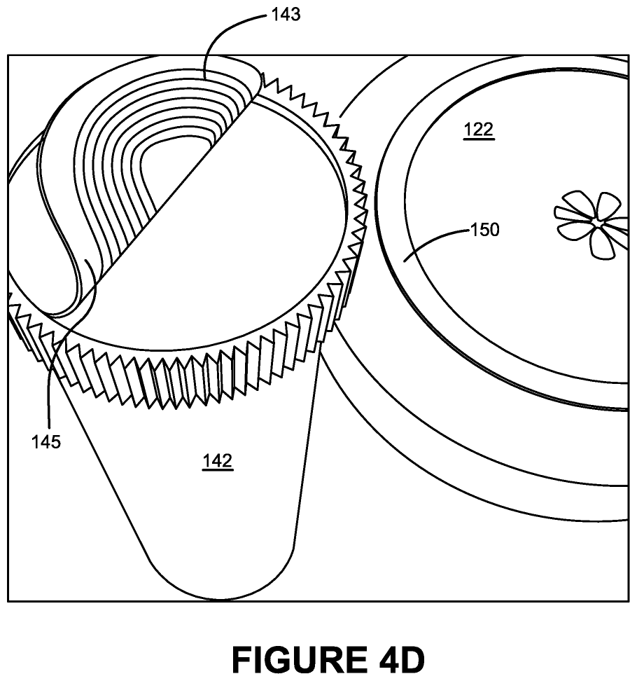

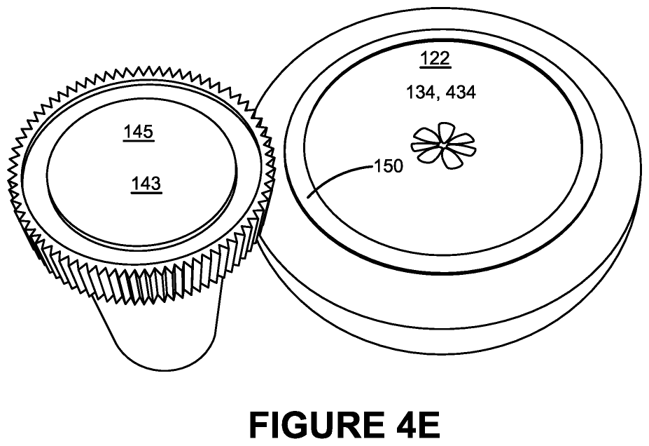

As shown in FIGS. 3, 4, 4A-4E, the docking station 122 may also comprise a visual indicator 150 operably connected to the base 124. The visual indicator 150 may be configured to provide visual feedback to the user. For example, visual feedback may comprise one or more of success for entering data (e.g. biometric entry of a student or nurse or administrator), success for matching data (e.g. identification of a student or nurse or administrator), failure of entering data, failure for matching data or status of an interaction for the user, such as a student nurse or administrator. Success, failure or completion may be indicated by different colors. For example, a successful action may be indicated with a green light. A red light may indicate an unsuccessful action. The light may blink intermittently. The light may appear to move to indicate a given interaction is pending. Any color may be chosen for any type of action. In one nonlimiting example, the visual indicator may be configured to be a light ring 152. The visual indicator 150 may be operably connected to any location of the base 124. In one nonlimiting example, the light ring 152 may be disposed about a perimeter 154 of the base 124. In another nonlimiting example, the light ring 152 may be disposed about a perimeter of the interaction portion 134.

The docking station 122 may further comprise a haptic indicator 170. The haptic indicator feedback may be a vibration or series of vibrations. For example, the haptic feedback may comprise one or more of success for entering data (e.g. biometric entry of a student or nurse or administrator), success for matching data (e.g. identification of a student or nurse or administrator), failure of entering data, failure for matching data or status of an interaction for the user, such as a student nurse or administrator. The haptic indicator may make different vibrations depending upon the action.

In one nonlimiting implementation, the docking station 122 may be a mobile docking station 422. The mobile docking station 422 may comprise similar configurations and features as the docking station 122 described above. The mobile docking station 422 may be utilized at an offsite event, such as on a field trip. With reference to FIG. 4, biometric information may be entered or read for a user, which may comprise a student, nurse, administrator or other authorized user during administration of medication. The docking station 422 may be utilized for identification of a student, authentication of who the student is, and unlocking an associated container. This may occur in an office setting. The docking station 422 may comprise a base 424, which may comprise an upper surface 426. The docking station may comprise a biometric reader 430. The biometric reader 430 may take any biometric input, including without limitation, fingerprints, iris recognition, retina recognition, palm veins, hand geometry, facial recognition, or DNA. In one nonlimiting implementation, the biometric read may be a fingerprint reader 432. The fingerprint reader 432 may be sized to receive at least one digit. In other implementations, it may be sized to receive two or more digits. In other implementations, in addition to or instead of the biometric reader, any other type of reader may be utilized to receive data from any type of scanning identification to identify a user or a student.

With reference to FIG. 4, the mobile docking station 422 may further comprise an interaction portion 434. The interaction portion 434 may also be disposed somewhere on the base 424. In one implementation, the interaction portion 434 may be disposed on the upper surface 426 of the base 424. The interaction portion may be configured to comprise a communication tag reader 436. The interaction portion may be configured to also comprise an unlocking mechanism 438. In one implementation, the unlocking mechanism 438 may comprise a magnetic array that transfers motion to a lock 750 operably connected to a container 142.

The mobile docking station 422 may also comprise an audible indicator 447 operably connected to the base 424. The audible indicator 447 may be configured to provide audible feedback to the user. In one nonlimiting example, the audible indicator may be a sound port 448. For example, the audible feedback may comprise one or more of success for entering data (e.g. biometric entry of a student or nurse or administrator), success for matching data (e.g. identification of a student or nurse or administrator), failure of entering data, failure for matching data or status of an interaction for the user, such as a student nurse or administrator. The audible indicator may make different sounds depending upon the action. By way of nonlimiting example, if the action is successful, the audible sound may be a higher pitch or a ding. However, if the action is unsuccessful, the audible sound may be a buzzer sound or other sound indicative of a negative result. A wide array of sounds may be chosen indicative of the status or action.

The mobile docking station 422 may also comprise a visual indicator 450 operably connected to the base 424. The visual indicator 450 may be configured to provide visual feedback to the user. For example, visual feedback may comprise one or more of success for entering data (e.g. biometric entry of a student or nurse or administrator), success for matching data (e.g. identification of a student or nurse or administrator), failure of entering data, failure for matching data or status of an interaction for the user, such as a student nurse or administrator. Success, failure or completion may be indicated by different colors. For example, a successful action may be indicated with a green light. An unsuccessful action may be indicated by a red light. The light may blink intermittently. The light may appear to move to indicate a given interaction is pending. Any color may be chosen for any type of action. In one nonlimiting example, the visual indicator may be configured to be a light ring 452. The visual indicator 450 may be operably connected to any location of the base 424. In one nonlimiting example, the light ring 452 may be disposed about a perimeter 454 of the base 424. In another nonlimiting example, the light ring 452 may be disposed about a perimeter of the interaction portion 434.

The mobile docking station 422 may be configured to have a wireless connection 460. A local area network (e.g., a short-range or nearfield communication network) can be established between mobile docking station 422 and the computing device 118. As an example, the mobile docking station 422 can comprise a Bluetooth module, and the computing device can comprise a separate Bluetooth module, which can be used to create a local Bluetooth connection with each other, for data sharing. The mobile docking station 422 may also comprise a charging port 462 to charge an on-board battery 464. Any on-board battery 464 may be chosen with sound engineering judgment, including without limitation, nickel cadmium batteries, nickel-metal hybrid batteries, lead acid batteries, lithium ion batteries, lithium polymer batteries, or alkaline batteries. A charging port 462 may also be disposed in the mobile docking station 422 to deliver charge to the on-board battery 464. The mobile docking station 422 may also comprise an accessory loop 466 operably connected to the base 424. The accessory loop 466 may be configured to be of sufficient length so that a nurse or administrator may position the accessory loop 466 over their head such that the accessory loop 466 may function as a lanyard. The accessory loop 466 may also be of sufficient size to be easily carried in the nurse or administrator's hand, wrist, or crux of an arm.

The mobile docking station 422 may further comprise a haptic indicator 470. The haptic indicator feedback may be a vibration or series of vibrations. For example, the haptic feedback may comprise one or more of success for entering data (e.g. biometric entry of a student or nurse or administrator), success for matching data (e.g. identification of a student or nurse or administrator), failure of entering data, failure for matching data or status of an interaction for the user, such as a student nurse or administrator. The haptic indicator may make different vibrations depending upon the action.

With reference to FIGS. 4A and 4B another implementation of a mobile docking station 422 is shown. The mobile docking station 422 may comprise an interaction portion. It may also comprise a visual indicator and audible indicator as previously described. The mobile docking station 422 may operably connected to a computer device 118. The mobile docking station 422 may be configured to have a wireless connection 460. A local area network (e.g., a short-range or nearfield communication network) can be established between mobile docking station 422 and the computing device 118. As an example, the mobile docking station 422 can comprise a Bluetooth module, and the computing device can comprise a separate Bluetooth module, which can be used to create a local Bluetooth connection with each other, for data sharing. The mobile docking station 422 may also comprise a charging port 462 to charge an on-board battery 464. Any on-board battery 464 may be chosen with sound engineering judgment, including without limitation, nickel cadmium batteries, nickel-metal hybrid batteries, lead acid batteries, lithium ion batteries, lithium polymer batteries, or alkaline batteries. A charging port 462 may also be disposed in the mobile docking station 422 to deliver charge to the on-board battery 464.

In a nonlimiting implementation, a sensor 143 may be utilized to operably communicate with the interaction portion 134, 434. The sensor, which may include without limitation, a near field communication (“NFC”) or radio frequency identification (“RFID”) sensor 143 may be disposed on the container 142, such as the bag 500, for association of the medication on the student record. The sensor may comprises medication data and/or student data as described herein. When the sensor 143 operatively communicates with the interaction portion 134, 434, data is read from the sensor and is sent to the computing device, for example. This may enable for verification of the person (such as the student) and the particular data associated with that student as well as particular medication data.

In another implementation, the NFC sensor or RFID sensor 143 may be disposed on the container 142, such as the pill bottle. With reference to FIGS. 4C, 4D, and 4E, in one implementation, the NFC sensor or RFID sensor 143 may be disposed or embedded in a sticker 145 having adhesive, so that the sticker with the sensor may adhere to the container 142, where as previously stated, can take the form of a bag, a storage bin, a tray, or a pill bottle or any other vessel suited to hold medication. For example, the RFID or NFC sensor 143 disposed on the sticker 145 may be disposed on the cap of a pill bottle.

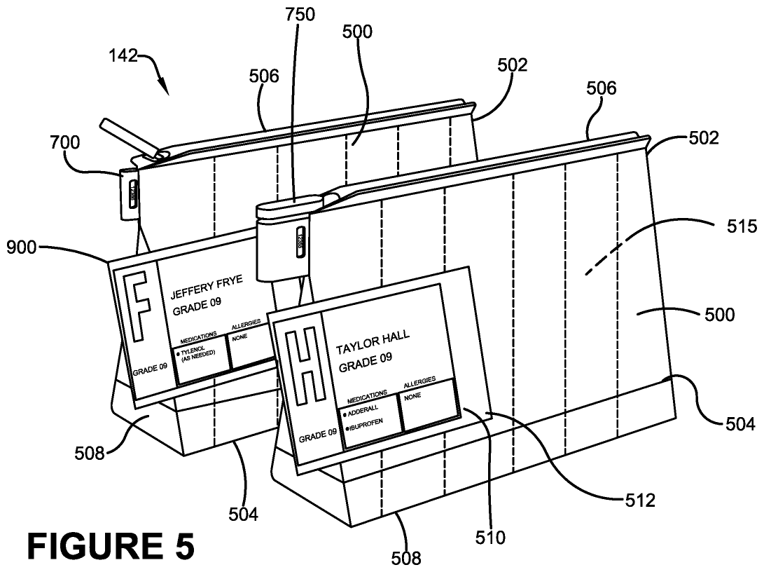

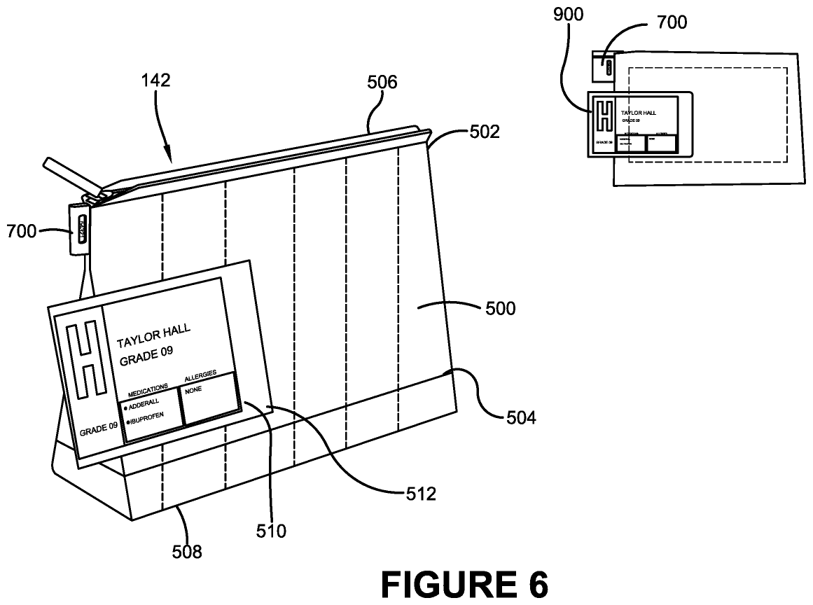

With reference to FIGS. 5 and 6, the container 142 is shown in more detail. The container 142 may take any form chosen with sound engineering judgment, including without limitation, a bag, a storage bin, a tray, or a pill bottle. In one implementation, the container 142 may have a lid or form of closure. The lid or closure may be lockable. The container 142 may manufactured from a soft material, such as a durable fabric or it may be made of a plastic material. In other examples, the container may be made of tamper resistant fabrics. In other implementations, the fabric may comprise metal strands woven through its bias. In a nonlimiting implantation, the container 142 may be a bag 500. The bag may be lockable or unlockable. The bag 500 may have an upper area 502 and a bottom area 504 oppositely disposed from the upper area 502. The bag may also have sides 503. The bag 500 may further comprise a zipper 506 disposed proximate to the upper area 502. The bag 500 may also comprise a base 508 of sufficient area such that the bag 500 may be positioned upright on a surface. In another example bag 500, the base 508 may be weighted to maintain the bag in an upright position. An inside of the bag is of sufficient dimension to hold desired contents, such as medication. One of the sides may have a pocket 510 disposed therein. In one implementation, the pocket 510 may be formed with one of the sides 503 of the bag 500 and a flexible member 512. The flexible member may be a clear plastic or other transparent material. The pocket may be sized and dimensioned to receive an identification card 900 of the student.

With continuing references to FIGS. 5 and 6, the bag 500 may also comprise a communication tag 700. Each bag 500 may have a unique communication tag 700 operably connected thereto to match a student with their needed medication. Matching with the student may occur during the authentication, administration and documentation process. A lock 750 may be utilized to secure the bag 500 whose contents may require additional security. A zipper closure 514 may interface with the lock 750 to secure the zipper 506. In another implementation, the lock 750 may not require special tools to access the medication or other contents of the bag 500.

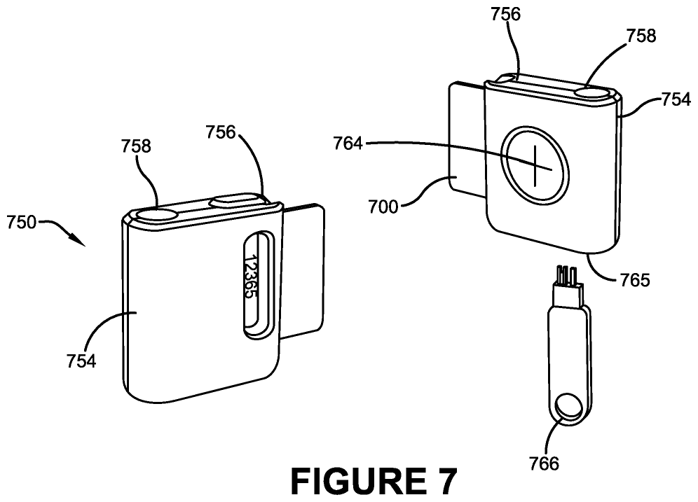

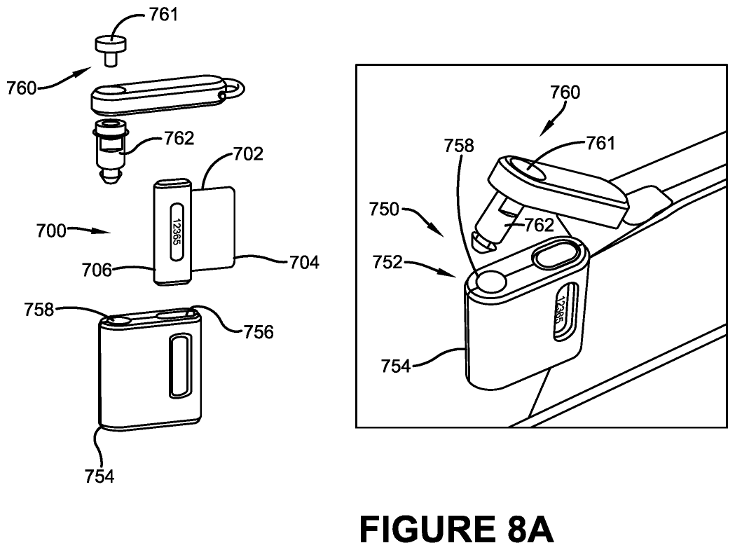

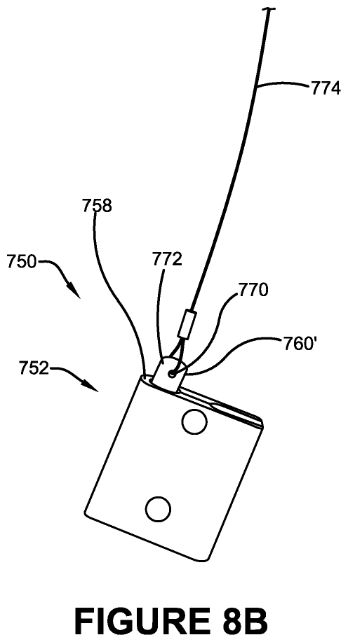

With reference to FIGS. 7, 7B and 8A, the lock 750 and communication tag 700 is further described. The communication tag 700 may be operatively connected to the bag 500. In one implementation, the communication tag 700 may be stitched to the bag 500 towards the upper area 502. The communication tag 700 may comprise a body 702 with an attachment portion 704 and a lock engagement portion 706. The attachment portion 704 may be a tab that is operably connected to the bag 500. The lock engagement portion 706 may comprise a unique identifier, such as a serial number, to correspond with a student. The unique identifier may be visible when the communication tag 700 may be operably connected to the bag 500.

The lock 750 may be a lock assembly 752. The lock assembly 752 may comprise a housing 754. The housing 754 may have a first opening 756 defined therein to engage with the communication tag 700. In one implementation, the lock engagement portion 706 may engage with the first opening 756 of the housing 754. In another implementation, the lock engagement portion 706 may be in selective and sliding engagement with the first opening 756. The housing 754 may have a second opening 758. The second opening 758 may be configured to receive a locking pin 760. The locking pin 760 may comprise a threaded cap 761 configured to selectably engage with a locking pin body 762. The locking pin 760 may pass through an opening 515 of the zipper closure 514. The locking pin 760 may be selectably slide into and out of the second opening 758. The housing 754 may further comprise an interface portion 764 disposed on one side of the housing 754. The interface portion 764 may mate or operably engage with the interaction portions 134, 434 of the docking station 122 and the mobile docking station 422. When the interface portion 764 mates or operably engages with the interaction portions 134, 434, the locking pin 760 may be released from the second opening 758, allowing the zipper closure 754 to move and unzip the zipper 506 to open the bag 500. The lock assembly 752 may not require power to operate. In one implementation, the lock assembly 752 may be utilize magnetic arrays.

With reference to FIG. 8B, another implementation of the lock assembly 752 is shown. A locking pin 760′ is shown at least partially disposed in the second opening 758. The locking pin 760′ may be partially disposed, mostly disposed or completely disposed within the second opening. The locking pin 760′ may have a hole 770 defined at an end 772. A cable 774 may be operably connected through the hole 770 and then operably connect to the opening 515 of the zipper closure 514. In one implementation, the cable 774 may be crimped, tied, or looped onto the opening 515 of the zipper closure 514.

The housing 754 may also comprise a key hole 765 located on a bottom surface of the housing 754. A safety key 766 may be configured to selectably engage with the key hole 765 to unlock the bag 500. In one implementation, the key hole 765 may have projections 768 to selectably engage with the matching recesses in the key hole 765. The safety key 766 may be utilized in the event the interaction portion 764 fails to unlock the lock assembly 752. Alternatively, the safety key 766 and key hole 765 may be utilized instead of the interaction portion 764 by pure choice of the user.

While the lock 750 has been described as a lock assembly 752, the lock 750 may be any other device chosen with sound engineering judgment. Lock 750 may be a padlock that passes through the zipper closure 514.

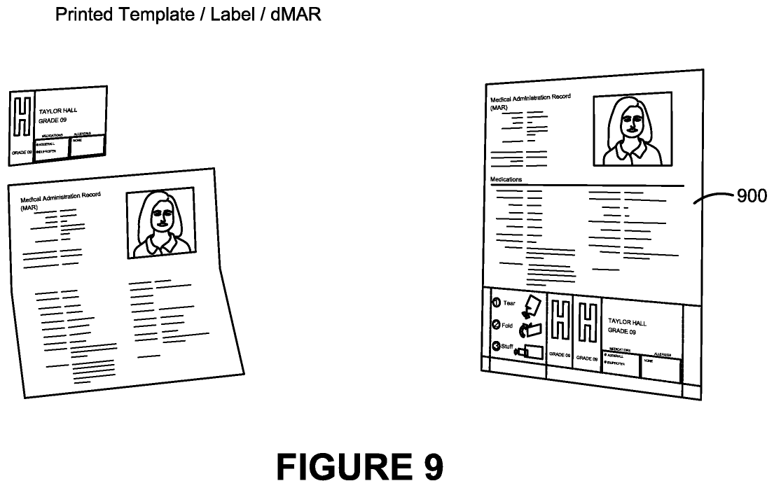

With reference to FIG. 9, the identification card 900 is described. The identification card may take the form of a digital medical administration record. Any student information may be provided on the identification card 900. The identification card may be configured to be inserted into the pocket 510 for easy display of student information. One portion of the identification card 900 may include an enlarged letter designating the first letter of the student's last name. The enlarged font may provided for swift and easy identification. Information that may be included, for example, may be the student's full name, grade, medications, and allergies. Information may be hidden from view as well to comply with privacy regulations. The information provide on the identification card 900 may comprise a portion or the entire record found in the system 100. As such, as information is updated, a new identification card 900 may be printed so the information contained thereon is current. The identification card 900 may also serve as a printed record of detailed information about the student, the student's medication, necessary instructions or other pertinent information. In one implementation, the identification card may provide the same information in the student record's record as inputted through the transactional information component 1002. As such, if medicine is administered off-site, the user may have all the same information as is found in the transactional information component 1002.

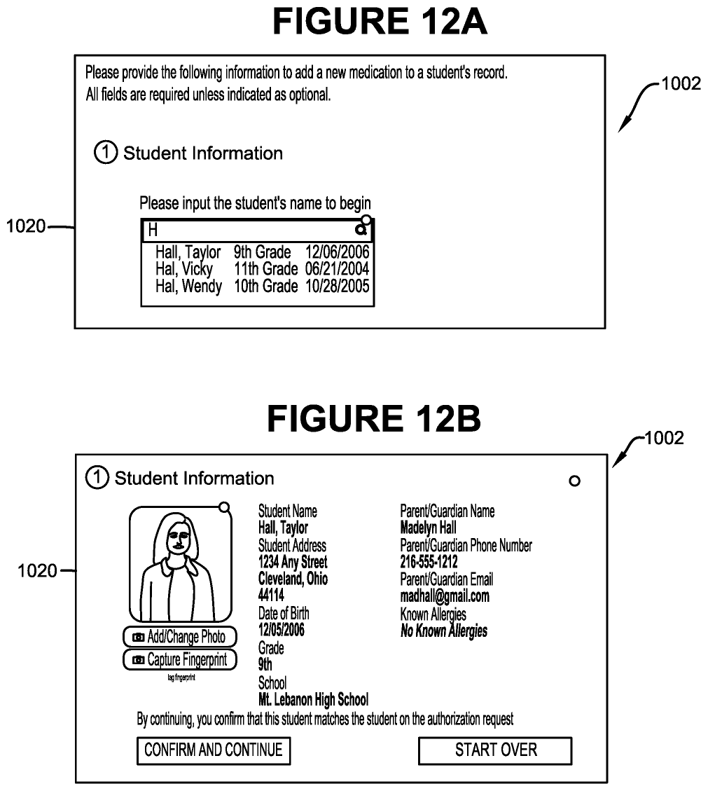

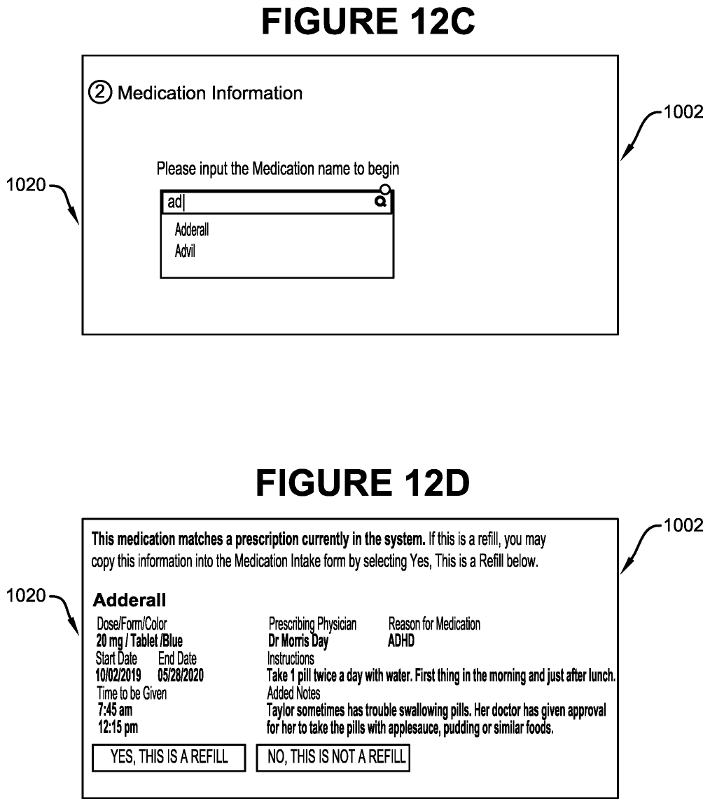

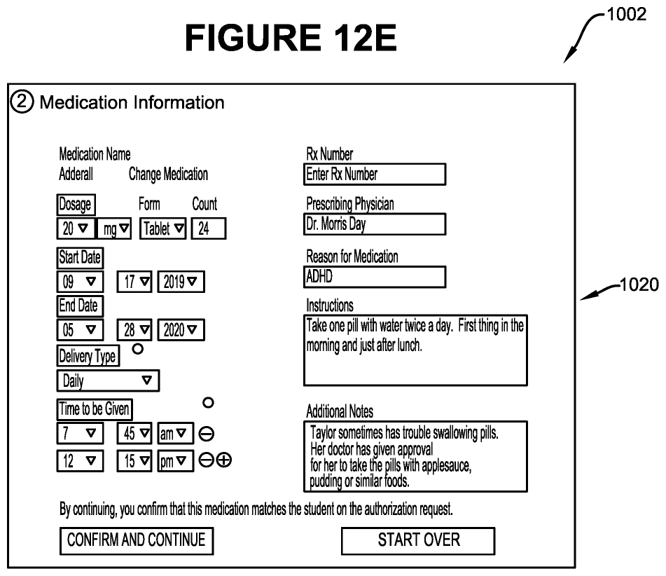

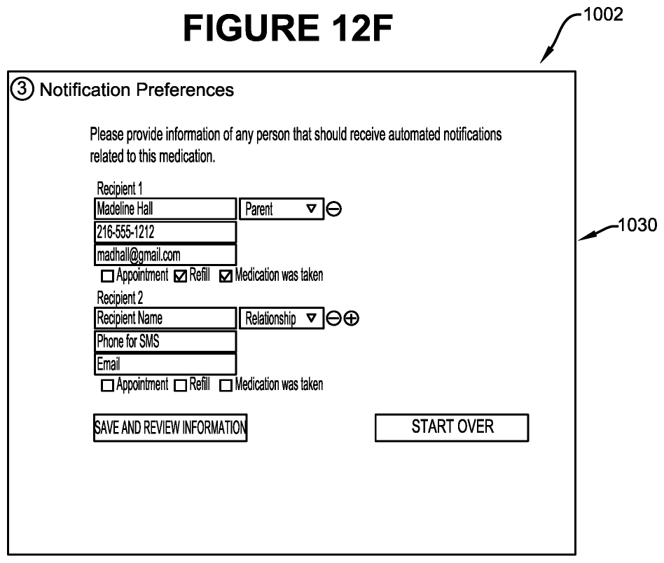

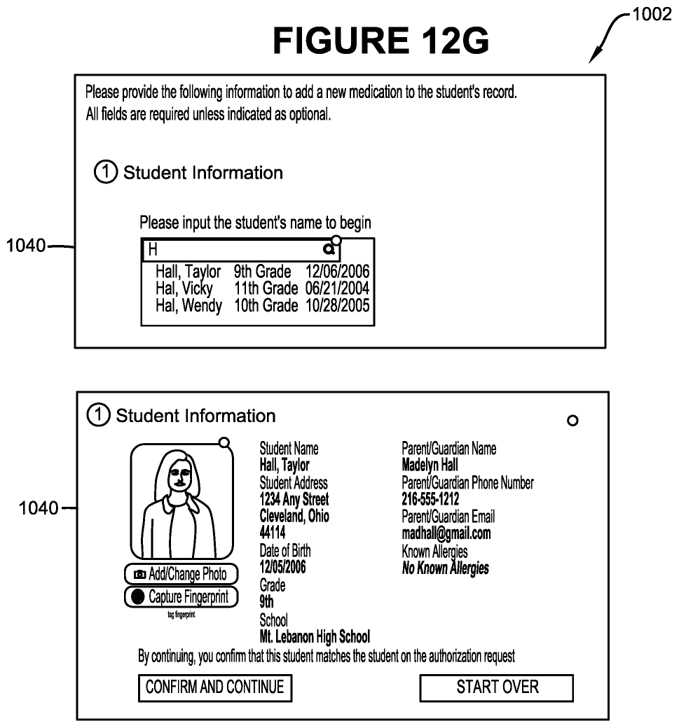

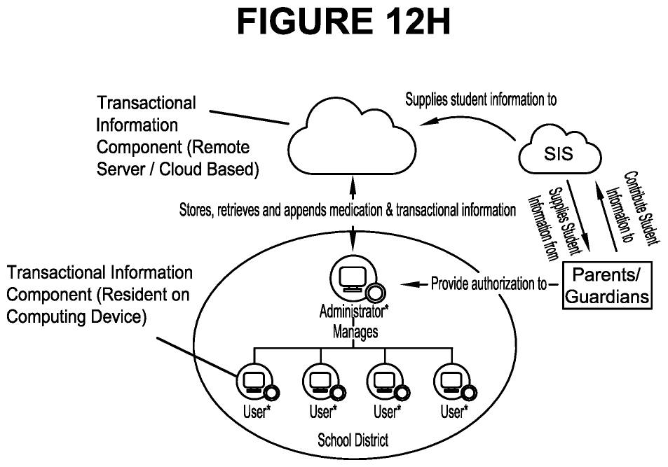

The medicinal compliance system may comprise a variety of components as described herein utilized to verify a person (e.g. student), an authorized user (e.g., nurse) and manage various records directed to medication taken by a given student. As shown in FIG. 12H, the medicinal compliance system may exchange data between by the transactional information components, which may be between the computing device and a remote server. The student information system also sends data to the transactional information system on the remote server. The transfer of data among the components can occur regardless with varying features of the docking station, container, locks, or sensors.

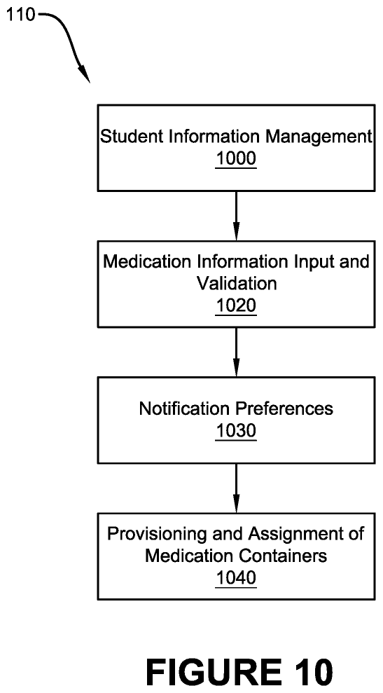

The new medication intake process 110 may provide the school nurse and/or administrator the ability to input new or updated prescription information into the medicinal dosage compliance system 100. This may enable association with a selected student. With reference to FIG. 10, the new medication intake process 110 may comprise student information management 1000, medication information input and validation 1020, notification preferences, such as email and sms messaging, 1030, and provisioning and assignment of medication containers 1040, such as the bag 500, previously described.

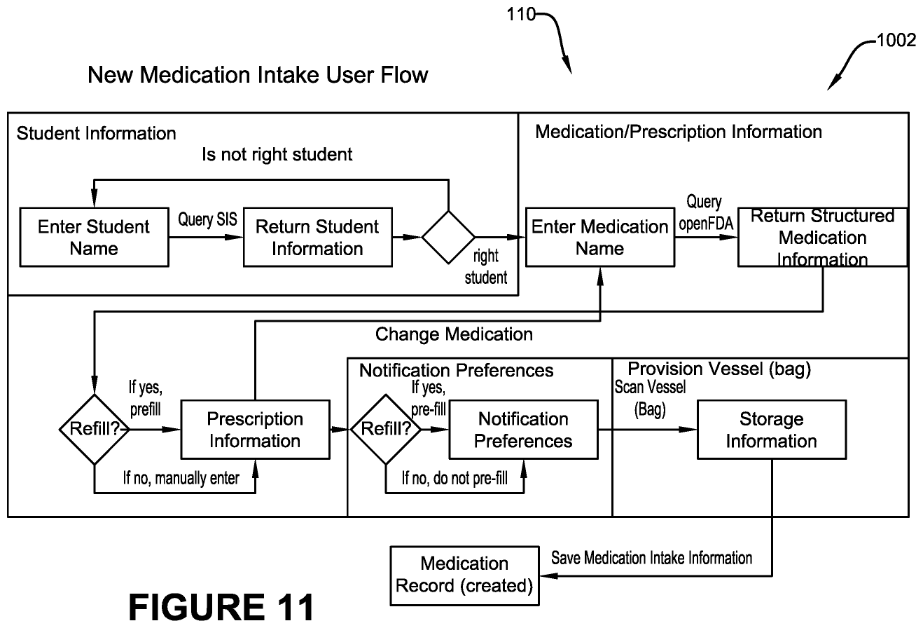

FIG. 11 illustrates the user flow for one example of the new medication intake process 110. Student information management 1000 may take place within a student information system and a transactional information component 1002. An example of a student information system may be a third party software program, such as ProgressBook owned by Software Systems, LLC. Likewise, the transactional information component 1002 may be a software system resident on a general computer or available through a remote server, such as a cloud application or other means, such as a mobile application. The transactional information component 1002 may receive medication data and person (e.g. student) data sent from a computing device, which may be indicative of a database of medication transaction records. The transactional information component 1002 may integrate with the student information system. In one implementation, student information management 1000 may solely take place within the student information system to ensure verification of accurate student information. The student information system may store relevant information of all students within a school district or a single school. With the transactional information component 1002 integrating with the student information system, consistency of student information across multiple information systems will be ensured. In one implementation, the student information system data may be associated with a student's biometric data, such as fingerprint data. The transactional information component 1002 may also comprise functionality to capture and manage student images, such as photographs.

With reference to FIGS. 12A and 12B, a user (nurse or administrator, for example) may desire to enter a new medication into a student record. The user may enter the name of the student. The student information system is queried and returns matched responses. If multiple students have the same or similar name, additional identifying information may be provided including grade and date of birth. Once the correct student is identified, the transactional information component returns relevant personal information of the student that is stored and managed in the student information system. The user is then able to initiate capture and management of biometric data, such as fingerprint data, associated with the student. This information may be stored as a data layer within the transactional information component 1002.

Next, the medication information input and validation 1020 may provide a mechanism to store medication and prescription information in a consistent and structured manner. The transactional information component 1002 may leverage structured data provided by an openFDA application data interface (“API”) to ensure the consistent entry of medication information as well as to make additional information about the medication available to the transactional information component 1002. Use of openFDA is but one nonlimiting example of a database that may be used for accuracy of medicinal data. Any structured database with reliable and accurate information from a third party may be implemented. In one implementation, the medication information input and validation 1020 may comprise the completion of a medication information form by pre-filling the form if it is likely that the prescription is a refill.

Turning to FIGS. 12C and 12D, medication intake is shown. FIG. 12A shows that as the user enters the name of the medication, openFDA or other program is queried to return matched response. For example, if “AD” is entered, options for medication may be Advil for Adderall. Medications may be matched by generic name or brand name. As shown in FIG. 12D, if the prescription appears to be a refill, the user may have the ability to choose to pre-fill a medication information form. If they choose that it is not a refill, the form will be presented with no information pre-filled. Prior to choosing that the prescription is a refill, the user may be asked to verify relevant identifying information related to the prescription.

With reference to FIG. 12E, a medication information form is shown. The medication form may comprise one or more fields for medication name, dosage, form, count, start date, end date, delivery type, time to be given, prescription number, prescribing health care provider, reason for the medication, instructions and additional notes. The user must may input relevant medication and prescription information into the form. Information in the form may be selectably updated. In one implementation, changing the medication name may clear the form and redirect the user back to input the medication name. In one implementation, the field for time to be given may be present if the delivery type is daily.

With reference to FIG. 12F, the step of notification preferences 1030 is shown. The transactional information component 1002 may provide a method to notify individuals of important events. In one implementation, parents and caregivers in the process can receive notifications by sms messages or email notifications. These notifications may comprise information related to medication refills, notification that the medication was taken, and medication reminders, such as it is time to take medication. As shown in FIG. 12F, the user may have the ability to set user preferences. This may further comprise the ability to configure automated messages of key events. Automated message configurations may comprise the ability to add and remove recipients from the list. It may also comprise the designation of a trigger for the notification, including without limitation, an appointment, a refill and that the medication was taken. If a user chooses to pre-populate the form as a refill earlier in the intake process, notification preferences from the previous prescription may be pre-filled. If the user does not choose to pre-populate the form, the form shown in FIG. 12F may be blank. The user may be able to edit notification settings from the prescription information provided in the student's digital medication administration record section.

The provisioning and assignment of medication containers 1040 is shown in FIG. 12G. The user may be prompted to scan the container 142, which may be the bag 500 as previously described. More specifically, the user may scan the bag 500 at either the communication tag 700 and/or the lock 750. In one implementation, the user's scan may be passing the interface portion 764 of the lock 750 that mates, passes over or otherwise operably engages with the interaction portions 134, 434 of the docking station 122 or the mobile docking station 422. After this action, the bag 500 may be associated with the medication or prescription record. The user may be asked to verify some or all information provided in the previous steps. Upon approval of the information, a record of the medication and/or prescription is created resulting in the recordation of the medication transaction to the digital medication administration record, which may be associated with the student. The medication transaction record itself or in combination with other student data or medication data may be an electronic medical record. The transactional information component 1002 may provide a method to store and secure medication upon completion of the intake process. Once the bag 500 is associated with the medication and/or prescription, this will enable the authentication process that may take place during the medication delivery process 112.

With reference to FIG. 12H, parents, physicians, or other third parties other than the user may access a web portal to view particular student data to whom they have authorized access. Reports may be generated from the student data. This may occur through the student information system, such as ProgressBook, or it could be through the transactional information component 1002 on a personal computer. The parents, physicians, or other third parties may have access to a dashboard for accessing the student's information, requesting prescription refills or providing messages. Authorization forms may be uploaded through the web portal to the transactional information component 1002. Student information, medication transaction records, and medication status may be accessed. This may also be the location for receiving the notifications previously described.

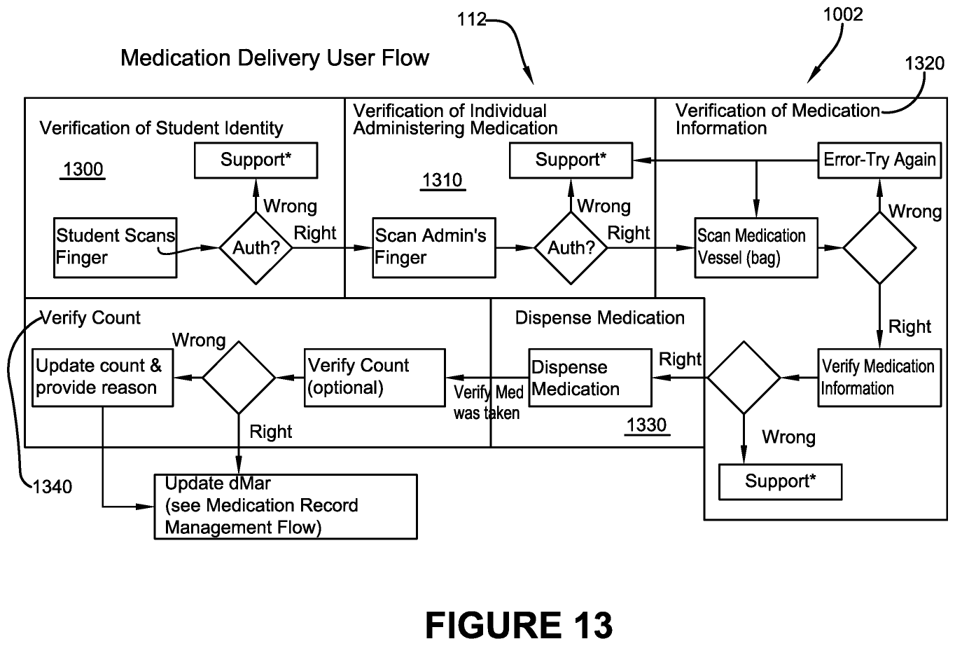

The medication delivery process 112 or a dispensing event may support the consistent and accurate delivery of medication to the student. Parameters, or medication data, to ensure compliance may include without limitation, the correct student, the correct medication, the correct dose, the correct time, and the correct route. With reference to FIG. 13, the medication delivery process 112 may include one or more of verification of student identity 1300, authentication of individual administrating medication 1310, verification of medication information 1320, dispense medication 1330, and verification of medication count 1340.

For verification of student identity 1300, the student may identify himself or herself by scanning a biometric marker, such as a fingerprint on the biometric reader 130, 430 of the docking station. In other implementations, the student may identify himself or herself simply by verbally announcing themselves to the user (such as a school nurse or administrator), and/or the user may recognize the student. Alternatively, the student may provide an identification card, such as a school issued student ID. The user verifies that the student's identity is correct and a record exists in the transactional information component 1102. The verification may occur through a visual and/or audio signal as previously described. The student's record may then be made available on a screen for the user to initiate the medication delivery process.

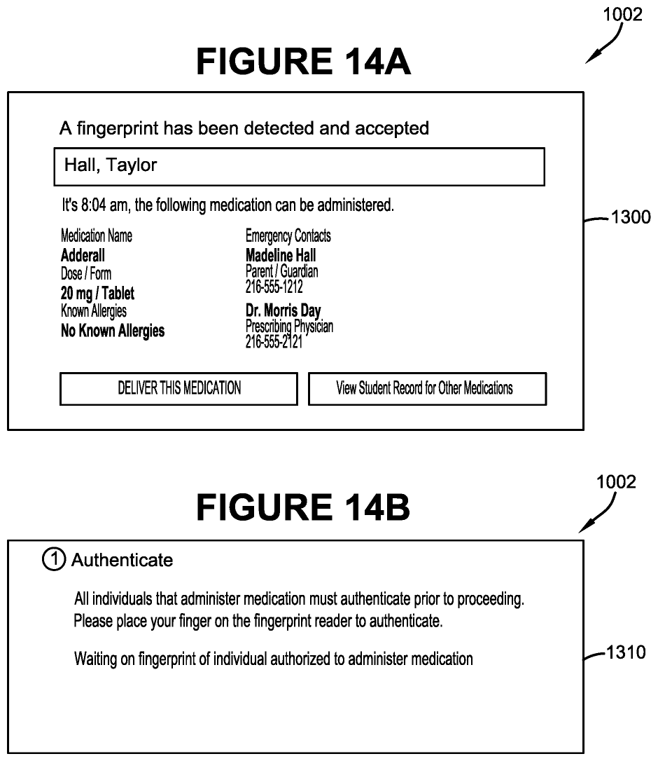

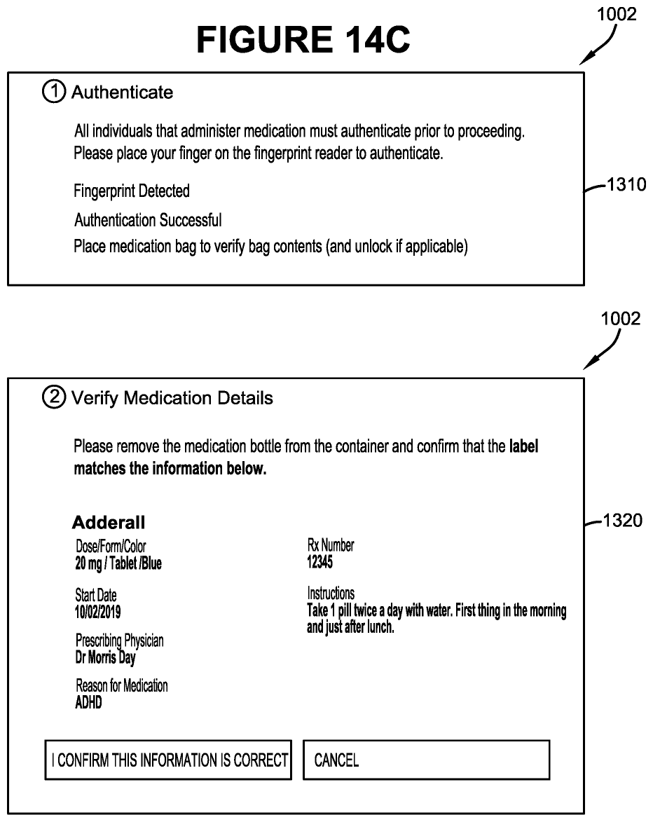

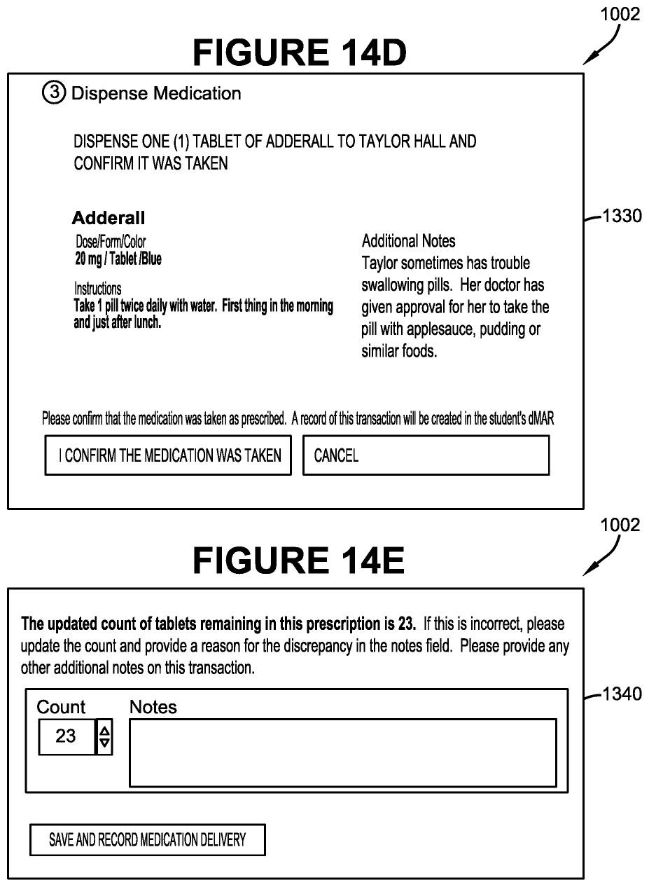

With reference to FIG. 14A, when the student scans their finger, a dialog box may open to acknowledge that the student is authenticated and presents information to the user that will help the user understand the nature of the medication to be delivered. In one implementation, an action of “deliver medication” may be selected, which enters the user into the medication delivery process 112. In one implementation, it is possible for the user to override the finger scan, such as in the event of an emergency or being off-site. It should be understood, that any biometric marker may be utilized to identify the student. As previously stated, nonlimiting examples of biometric markers may include fingerprints, iris recognition, retina recognition, palm veins, hand geometry, facial recognition, or DNA.

With reference to FIG. 14B, the identity of the user (nurse or administrator) administering medication 1310 is authenticated. The user verifies his or her identity by placing their finger on the biometric reader 130, 430 of the docking station 122, 422. In one implementation, it is possible for the user to override the finger scan, such as in the event of an emergency or being off-site. The user is authenticated to achieve validation that he or she has the rights to administer medication, providing a digital signature on the medication transaction, and enabling the scanning and/or unlocking of the container 142, such as the bag 500. When the user scans their fingerprint, the transactional information component 1002 may authenticate them, which enables them to access the medication inside the student's corresponding container 142. In one implementation, the user may be prompted with directions to authenticate their identity by placing his or her finger on the biometric reader 130, 430. In one implementation, if the user is not authenticated, the user must register in the transactional information component 1002 as an authorized user or the transactional information component 1002 will not proceed further. In some implementations with locked containers 142, the user would not gain access to the medication.

Next, verification of the medication information 1320 occurs. One nonlimiting implementation of screen prompt is illustrated in FIG. 14C. The user may scan the container 142 to identify the medication as the correct medication to be administered to the student. If the container 142 comprises a lock 750, the lock 750 may open when the user and the student are verified. The user may visually verify that the medication is correct prior to dispensing the medication to the student.

With reference to 14D, dispensing of medication 1330 may occur. The user may be prompted to dispense the medication. Medication information may be made available for cross reference at the time of administering the medication. The user may be asked to confirm that the medication was taken. At this point in the process, dual authentication occurs because the correct student has been identified and the user witnesses the student taking the medication.

FIG. 14E illustrates one implementation of verification of the medication count 1440. The user may verify the count of medication. For example, the user may be given the option to change the count of the medication after the student takes its prescribed dosage. If the count is changed, the user may be required to provide a reason in a field for notes or other designation. If the count does not change, the user may enter a note at his or her discretion.

The digital medication record management process 114 may provide access to all information related to the student's medication history in the transactional information component 1002. In some implementations, this may include management of each medication prescribed to the student as well as the history of medication transactions. The digital medication record management process 114 may be supported by new prescription intake information previously described and the medication delivery transaction previously described. In other implementations, vaccine administration and compliance may also be utilized.

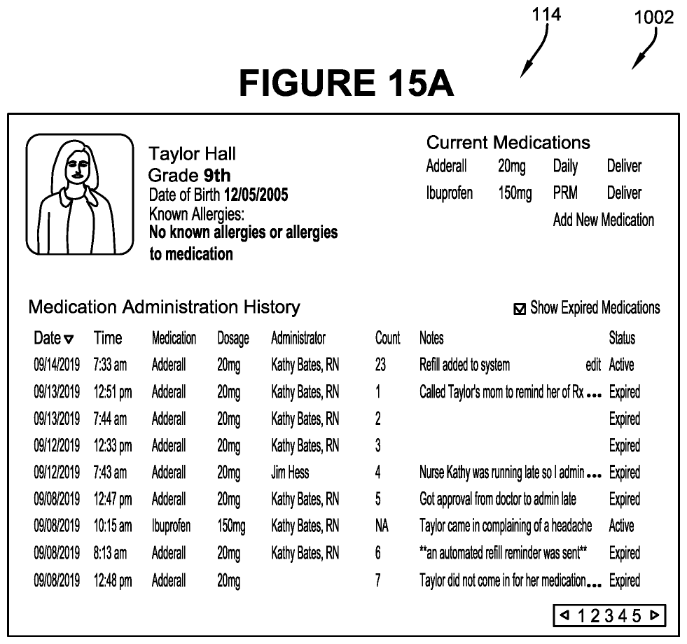

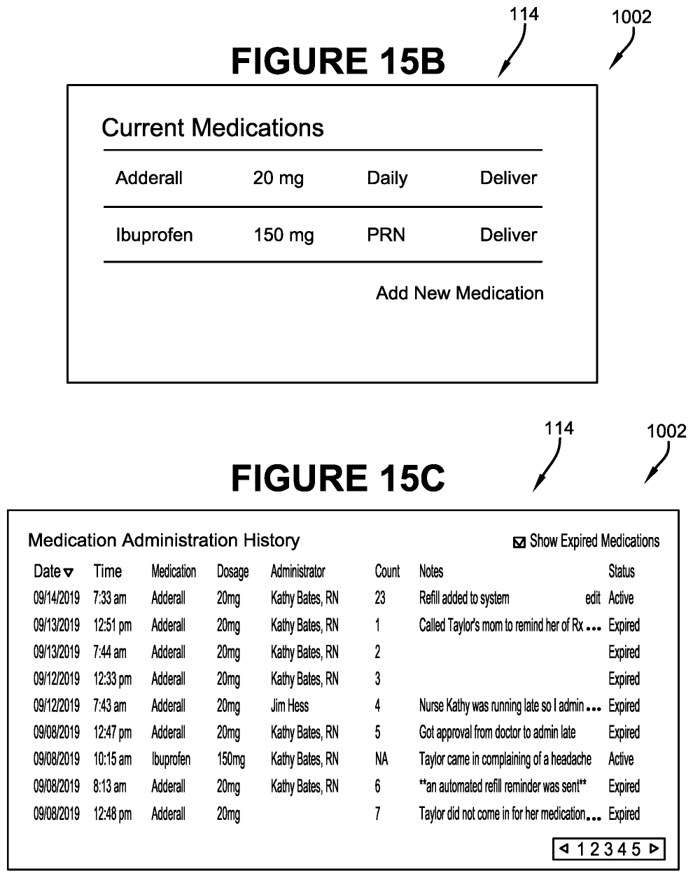

Turning to FIG. 15A, the digital medication record management process 114 is further described. An example of the digital medication administration record is shown. A current medication list may be provided as well as the student's identification information. A list of medication administration history may also be shown, which includes at least a portion or all of the medication transactions. In some implementations, the medication transaction history may be sorted by date or type of medication. As shown in FIG. 15B, a list of active medications may be displayed associated with an identified student. In one implementation, this may be the point of entering medication that was administered PRN (as needed) or for emergency reasons, such as a rescue inhaler for asthma. This may also be utilized as a secondary method to enter new medication.

With reference to FIG. 15B, an example of a screen view of a student's medication transactions through the medication administration history. The user may be able to view previous medication transaction with the student and associated data with each medication transaction. This may include who the user was, the medication name, dosage, and notes. Medication count and status may also be shown. An authenticated user with a sufficient level of access right can update the count and/or notes for the last transaction for a medication. In one implementation, no user can update medication transaction records further back than the last transaction of the medication. In this implementation, previous mistakes cannot be overwritten, which in turn, ensures accuracy of the digital medication administration record. The digital medication administration record may be resident on the computing device 118 and/or resident on a remote server as part the transactional information component 1002.

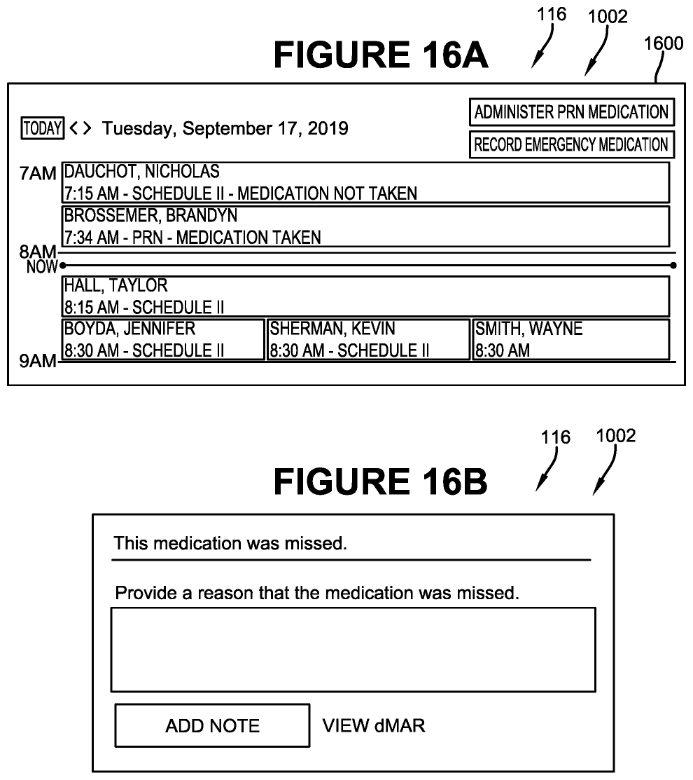

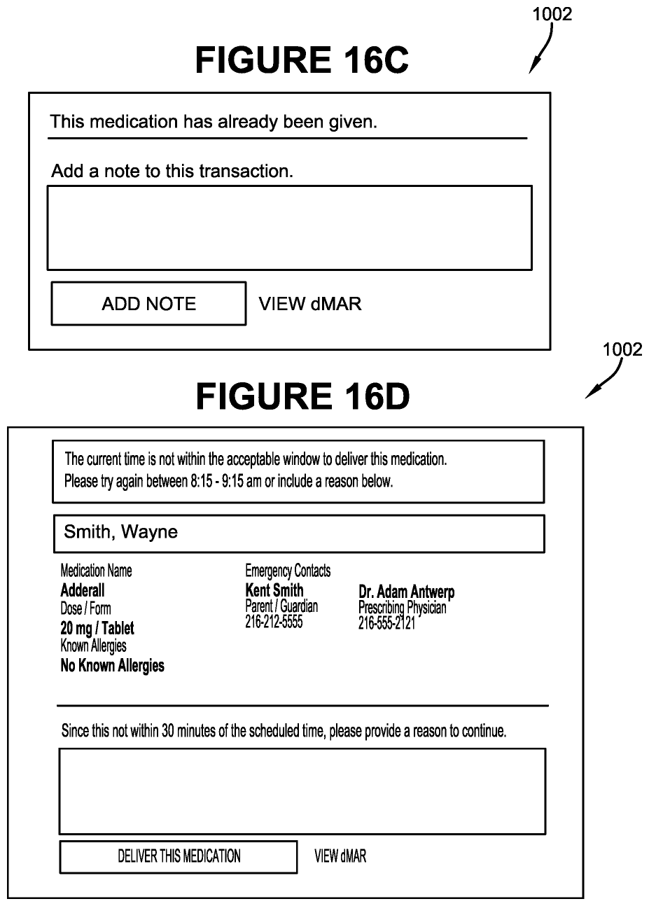

The scheduling process 116 may provide a centralized view of upcoming appointments, such as medication transactions, that the user may have with students. The scheduling process 116 may include a dashboard 1600, as shown in FIGS. 16A-16D. Functionality of the dashboard 1600 may assist the user can see an overview of the day. The dashboard 1600 may comprise an appointment time, which may be set during the student intake process. The dashboard 1600 may also provide a window of time that the medication can be administered. For example, the window may be about thirty minutes. In some implementations, the user may be permitted to administer medication outside the window time period. If this occurs, the user may include a note as part of the medical transaction.

With continuing reference to FIG. 16A, one implementation of the dashboard 1600 is shown. In one example, this may be a schedule. FIG. 16A illustrates all scheduled daily medications associated with a student. In one example, each appointment may be coded with visual indicators, such as colors and/or graphics. Further, the visual indicators may indicate the status of that medication, such as if it was taken (green) or missed (red). Another visual indicator may show an upcoming appointment (blue). In one implementation, selecting on of the appointments may display high-level information on the appointment and the student and provide a way to initiate the medication administration process. The user can also initiate manual administration of PRN and/or emergency medications. In another implementation, PRN medication transactions may be entered using the same flow as daily medications. In another example, administration of emergency medications may follow a variation of the flow that may enable administration with scanning the student's biometric data, such as their fingerprint.

FIGS. 16B and 16C illustrate the status of a scheduled medication. FIG. 16B indicate that the medication was scheduled to be delivered, but it was not. The user can still administer the medication, but it would be administered through the student's digital medical authorization record. The user can add a note to give a reason why the medication was missed. FIG. 16C indicates that medication was already given. The user can add a note to medication transactions successfully completed directly from the dashboard 1600.

With reference to FIG. 16D, the user can administer medication outside of the window period of time by selecting the student appointment. The user may provide a reason for administering the medication early to be able to proceed.

With use of the medicinal dosage compliance system, compliance with paperwork and medication schedules is streamlined. For example, for a given day at school setting, the user may come to his or her office and view the dashboard 1600 of students to be seen during the day, who need medication. For example, a first student may arrive at the user's office. The student places his or her finger on the fingerprint reader, which may be authenticated by the transactional information component 1002. The user places their finger on the finger print reader to authenticate they have administration rights. The user then finds the appropriate container 142, which is scanned on the docking station. If necessary, the container may be unlocked once it is matched in the system. By scanning the container 142 (e.g., NFC or RFID sensor), the transactional information component 1002 associates container with the student, and the user can ensure the match. The student can then take the correct dosage of the correct medication at the correct time. Afterwards, the user may authenticate that the student took the medication by either entering it manually into the transactional information component 1002 or by placing his or her finger on the fingerprint reader. The medication transaction is recorded in the student's digital medication authorization record. Over time, refill request may be automatically sent to a prescriber's office to assist with timely refills and no need of extra paperwork. Automated medication transaction records may be recorded. These records over time may be utilized through school compliance audits to ensure federal, state, and local laws are being met. Data found in the medicinal dosage compliance system 100 may be filtered in any manner. For example, medication transaction data may be filtered to generate a report to show compliance with an audit. The data may be filtered by grade, school building, age, date, types of medication, percentages of medicine taken within compliance parameters, or percentages of medicine taken outside of compliance parameters. In one implementation, reports may be generated through a web portal 1780 by a parent or guardian or physician. In another implementation, reports may be generated by the user through the system resident at the school location.

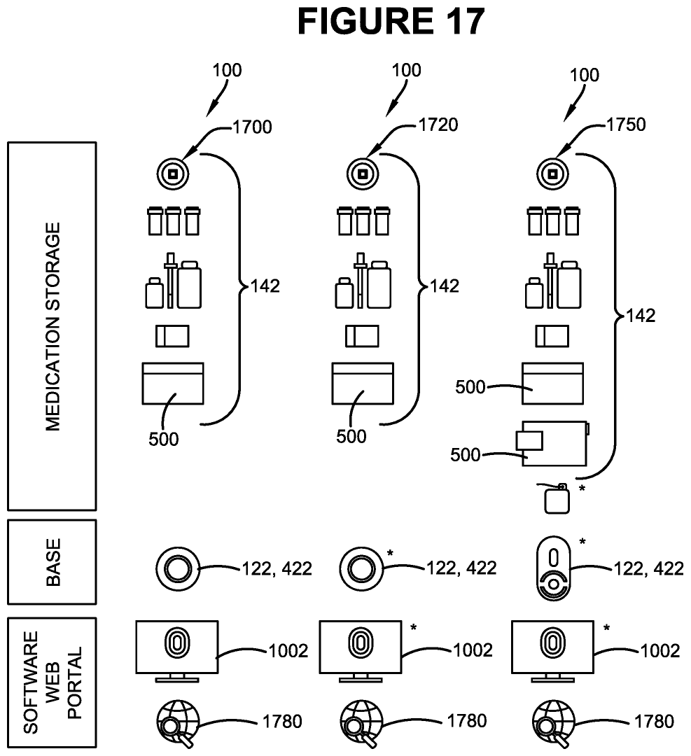

With reference to FIG. 17, a variety of implementations of the medicinal dosage compliance system 100 is shown. System 1700 may comprise containers, docking station, the software system used at the school by the user and the web portal 1780 access for parents, guardians and health care providers. The system 1700 may only authenticate the container through scanning a sensor, such as an RFID or NFC tag. Another system 1720 may add a security feature to the biometric reader to verify the identity of the student and the user in order to gain access to the medication found in a container as previously described. System 1750 may incorporate the features of systems 1700 and 1720, and add the additional feature of the lock assembly on the container. It should be understood that various features may be selectively added or removed to accomplish the required level of compliance for taking medication.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A dosage compliance system, comprising:
a docking station comprising an interaction portion;
a container configured to hold medicine for a person; and
a transactional information component in communicatively coupled with a computing device, wherein the person is identified, the container is identified to be associated with the identified person, medication data is communicated to the transactional information component indicative of a dosing event.

2. The dosage compliance system of claim 1, wherein the container further comprises a sensor configured to operably engage with the interaction portion of the docking station.

3. The dosage compliance system of claim 2, wherein the sensor is one or more of an RFID sensor or an NFC sensor.

4. The dosage compliance system of claim 1, further comprising a student information system is communicatively coupled with the transactional information component, the student information system comprising student data about the identified person.

5. The dosage compliance system of claim 4, wherein the information system is a student information system, the identified person is a student, the student information system configured to receive data about the identified student from a parent or guardian, a user being identified to access the transactional information component and the computing device, the user being a health care provider.

6. The dosage compliance system of claim 5, wherein the transactional information component is resident on a computing device.

7. The dosage compliance system of 5, wherein the transactional information component is resident on a remote server.

8. The dosage compliance system of claim 1, wherein the transactional information component is configured to have a user confirm the identity of the person and send data into the transactional information component to confirm the dosage amount of medicine taken by the identified person and a time when the medicine was taken by the identified person.

9. The dosage compliance system of claim 1, the docking station further comprising a biometric reader, wherein the person intake system is configured to have the user confirm the identity of the person with the biometric reader, and the user sends data into the transactional information component to confirm the dosage amount of medicine taken by the identified person and a time when the medicine was taken by the identified person.

10. The dosage compliance system of claim 1, wherein the container is one or more of a bag, a storage bin, a tray, or a pill bottle.

11. The dosage compliance system of claim 1, further comprising a lock configured to secure the container in a closed position.

12. The dosage compliance system of claim 1, further comprising a digital medical administration record for the identified person configured to record medication transactions over time.

13. The dosage compliance system of claim 1, wherein the docking station is a mobile docking station.

14. The dosage compliance system of claim 1, wherein the docking station comprises one or more of an audible indicator and a visual indicator.

15. The dosage compliance system of claim 1, wherein the medication is one or more of pills, liquid medication, and vaccines.

16. A dosage compliance system, comprising:
a docking station comprising an interaction portion;
a lockable container configured to hold medicine for a student, the lockable container configured to operably engage with the interaction portion;
a transactional information component in operative communication with a computing device accessible by a user, wherein the student is identified, the lockable container is identified and associated with the identified student, medication data is communicated to the transactional information component indicative of a dosing event, the medication data comprising at least a type of medication, a dosage amount of medicine to be taken by the student and a time when the medicine was taken by the student; and
a digital medical administration record for the identified person configured to record the medication data;
wherein upon the lockable container engages with the interaction portion, the container unlocks for the dispensing event and sends student identification data and medication data to the computing device.

17. The dosage compliance system of claim 16, wherein the lockable container further comprises a bag, the bag configured to hold medication, the lockable container further comprising a student identification card, the student identification card comprising student data.

18. The dosage compliance system of claim 16, wherein the docking station further comprises one or more of a biometric reader configured to confirm identity of the student and the user, the docking station comprising one or more of an audible indicator and a visual indicator.

19. The dosage compliance system of claim 16, further comprising a student information system communicatively coupled with the transactional information component, the student information system accessible by a parent or guardian of the student.

20. A dosage compliance system, comprising:
a docking station comprising an interaction portion;
a lockable container configured to hold medicine for a student, the lockable container configured to operably engage with the interaction portion, wherein the container is one or more of a bag, a storage bin, a tray, or a pill bottle;
a transactional information component in operative communication with a computing device accessible by a user, wherein the student is identified, the lockable container is identified and associated with the identified student, medication data is communicated to the transactional information component indicative of a dosing event, the medication data comprising at least a type of medication, a dosage amount of medicine to be taken by the student and a time when the medicine was taken by the student;
a student information system communicatively coupled with the transactional information component, the student information system accessible by a parent or guardian of the student;
a digital medical administration record for the identified person configured to record the medication data;
wherein upon the lockable container engages with the interaction portion, the container unlocks for the dispensing event and sends student identification data and medication data to the computing device.
